(19) United States
(12) Patent Application Publication
(10) Pub. No.: US 2019/0133968 A1
(43) Pub. Date: May 9, 2019

(54) Applications of benzophenones in preparation of drug

(71) Applicants: SHANXI MEDICAL UNIVERSITY, Taiyuan, Shanxi (CN); SHANXI MEDICAL UNIVERSITY OF TRADITIONAL CHINESE MEDICINE, Jinzhong, Shanxi (CN)

(72) Inventors: Qingshan Li, Jinzhong, Shanxi (CN); Xiue Feng, Taiyuan, Shanxi (CN); Yuanlin Zhang, Taiyuan, Shanxi (CN); Lingge Han, Taiyuan, Shanxi (CN); Rongrong Chang, Taiyuan, Shanxi (CN)

(21) Appl. No.: 16/097,348

(22) PCT Filed: Apr. 27, 2017
(86) PCT No.: PCT/CN2017/082173

§ 371 Date: Oct. 29, 2018

(30) Foreign Application Priority Data
Apr. 29, 2016 (CN) 201610278158.4
Apr. 25, 2017 (CN) 201710278835.7

(51) Int. Cl. A61K 31/12; A61K 31/5377; A61P 13/12

(52) U.S. Cl. CPC A61K 31/12; A61K 47/26; A61P 13/12; A61K 31/5377

(57) ABSTRACT

Medical applications of 2,5′-dibromo-4,5,2′-trihydroxyldiphenylmethanone and 4,5,2′-tris(4-morpholinemethanoyl)-2,5′-dichlorobenzophenone are provided, particularly applications in preparation of drugs respectively for resisting type II diabetic nephropathy and for treating acute pyelonephritis and chronic nephritis. Moreover, two drugs respectively with the above two compounds as the active ingredient are provided, respectively for resisting the type II diabetic nephropathy and for treating the acute pyelonephritis and the chronic nephritis. Both of the two compounds are able to effectively improve renal functions of model rats suffered from the type II diabetic nephropathy, the acute pyelonephritis, and the chronic nephritis, showing an important application prospect in resisting the diabetic nephropathy and treating the acute pyelonephritis and the chronic nephritis.

### CROSS REFERENCE OF RELATED APPLICATION

This is a U.S. National Stage under 35 U.S.C 371 of the International Application PCT/CN2017/082173, filed Apr. 27, 2017, which claims priority under 35 U.S.C. 119(a-d) to CN 201610278158.4, filed Apr. 29, 2016, and CN 201710278835.7, filed Apr. 25, 2017.

### BACKGROUND OF THE PRESENT INVENTION

### Field of Invention

The present invention belongs to a field of compound application and relates to two compounds which are able to prevent and treat type II diabetic nephropathy, acute pyelonephritis and chronic nephritis, specifically for applications of 2,5′-dibromo-4,5,2′-trihydroxyldiphenylmethanone and 4,5,2′-tris(4-morpholinemethanoyl)-2,5′-dichlorobenzophenone in preparation of drugs for preventing and treating the type II diabetic nephropathy, the acute pyelonephritis and the chronic nephritis.

### Description of Related Arts

The diabetic nephropathy (DN) is one of the common major complications of diabetes and will finally cause the end-stage renal disease (ESRD). At present, there are 114 million diabetics and 140 million pre-diabetics in China. With the continuously increasing diabetics around the world, the morbidity rate of DN increases. DN is the leading cause of ESRD in western countries, accounting for 25-42%, and is also the main cause of ESRD in China. The morbidity rate of DN in the type II diabetic nephropathy is about 20-25%. DN will firstly be found the appearance of urinary protein, further cause to hypertension and nephrotic syndrome, and finally lead to the kidney failure and death.

At the present stage, DN is treated mainly through controlling the blood glucose level, decreasing the blood lipids, inhibiting the inflammation, and resisting the oxidative stress. At present, for the clinical medication, the angiotensin converting enzyme inhibitor/angiotensin receptor blocker (ACEI/ARB) is mainly adopted and is even combined with the anti-diabetic drugs. The main functions of ACEI/ARB are to decrease the glomerular osmotic pressure through decreasing the blood pressure and microalbuminuria and to improve the insulin action. However, for most of the DN patients, especially the DN patients after phase IV, the application of the drugs in the class of ACEI/ARB will cause the electrolyte imbalance, which is easily combined with the hyperkalemia, and will lead to the side effects such as dry cough and orthostatic hypotension.

The acute pyelonephritis is the inflammatory disease of renal tubules, renal parenchyma, and renal parenchyma, which is mainly caused by the invasion of various pathogenic bacteria, while the part of glomerulus is generally less damaged. If the treatment is delayed, the acute pyelonephritis will turn into the chronic pyelonephritis, and finally develop into the kidney failure. The pathogenesis of the acute pyelonephritis mainly relates to the aspects of bacterial pathogenecity, inflammation and immunoreactions, and the latter two aspects have gained more and more attention from the researchers. At present, the appropriate antimicrobial drugs are mainly clinically selected for treating the acute pyelonephritis. However, with the abuse of the antimicrobial drugs, the drug-resistant strains continuously appear, and the acute pyelonephritis is easy to relapse after treatment, which affects the clinical curative effects thereof.

The chronic nephritis is the abbreviation of chronic glomerulonephritis, which is the immune-mediated inflammatory disease; the clinical manifestations comprise proteinuria and hematuria, and the patients may have different degrees of renal function decrease; the chronic nephritis refers to a group of glomerular diseases which have the renal function deterioration trend and will finally develop into the chronic kidney failure. In recent years, the incidence rate of the chronic nephritis gradually increases, and the patients tend to be younger, which brings great financial burden to families and societies. The recent study shows that the urinary protein is not only the marker of the glomerular diseases, but also the risk factor of the disease progression of the chronic and progressive kidney failure patients. A great number of clinical and experimental researches find that the urinary protein has the renal toxicity and will facilitate the kidney failure. Thus, decreasing and eliminating the urinary protein is one of the important measures for treating the chronic nephritis. At present, the classic hormone and immunosuppressant treatment is generally adopted for treating the chronic nephritis. Although the treatment has certain curative effect, the treatment is also accompanied by serious side effects and recurrence, so it is difficult to fundamentally avoid the development of the chronic nephritis.

Therefore, for the damages of the above three kidney diseases and the defects existing in the clinical drug treatment of the kidney diseases, finding the appropriate therapeutic drugs has become the research focus in the drug industry.

### SUMMARY OF THE PRESENT INVENTION

In order to solve problems in existing treatments of type II diabetic nephropathy, acute pyelonephritis and chronic nephritis, an object of the present invention is to provide pharmacological applications of 2,5′-dibromo-4,5,2′-trihydroxyldiphenylmethanone and 4,5,2′-tris(4-morpholinemethanoyl)-2,5′-dichlorobenzophenone, specifically for applications in preparation of drugs respectively for resisting the type II diabetic nephropathy and for treating the acute pyelonephritis and the chronic nephritis.

The other object of the present invention is to provide the drugs respectively for resisting the type II diabetic nephropathy and for treating the acute pyelonephritis and the chronic nephritis. The drugs adopt the 2,5′-dibromo-4,5,2′-trihydroxyldiphenylmethanone or the 4,5,2′-tris(4-morpholinemethanoyl)-2,5′-dichlorobenzophenone as an active ingredient, and further comprise an ordinary medicinal carrier or an excipient which is used combined with the active ingredient. According to different requirements, the drugs can be prepared into common dosage forms, such as solid dispersions, tablets, pills, capsules and injections.

Preferably, the compound of 2,5′-dibromo-4,5,2′-trihydroxyldiphenylmethanone (LM49) has a following structure of:

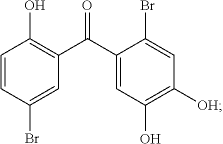 [SMILES: O=C(c1cc(Br)ccc1O)c1cc(O)c(O)cc1Br]

the compound of 4,5,2′-tris(4-morpholinemethanoyl)-2,5′-dichlorobenzophenone (A8) has a following structure of:

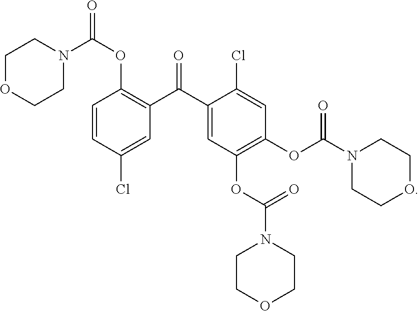 [SMILES: O=C(c1cc(OC(=O)N2CCOCC2)c(OC(=O)N2CCOCC2)cc1Cl)c1cc(Cl)ccc1OC(=O)N1CCOCC1]

The compounds of 2,5′-dibromo-4,5,2′-trihydroxyldiphenylmethanone and 4,5,2′-tris(4-morpholinemethanoyl)-2,5′-dichlorobenzophenone are two benzophenone compounds well known by one skilled in the art, and preparation methods thereof are easily realized.

According to the present invention, SD (Sprague Dawley) rat models of type II diabetic nephropathy, acute pyelonephritis and chronic nephritis are respectively established and administrated with the solid dispersions of the above two compounds of different doses by gavage; various indexes are measured during this period, such as blood glucose, blood lipids, urine volume, urine, visceral indexes, bacteria in the urine and tissues, related indexes of renal function, CD₄ and CD₈ in blood, and inflammatory factors, proving that the two compounds have strong kidney protective effects on the rats suffered from the type II diabetic nephropathy, the acute pyelonephritis and the chronic nephritis, and showing important application prospects of the two compounds in preventing and treating the type II diabetic nephropathy, the acute pyelonephritis and the chronic nephritis.

Main innovation points of the present invention are described as follows. Firstly, the two compounds are found to have an obvious pharmacological activity in resisting the type II diabetic nephropathy and an obvious pharmacological activity in treating the acute pyelonephritis and the chronic nephritis, and have good development and application values. Secondly, the two compounds have strong hypoglycemic, hypolipidemic, anti-oxidative and anti-inflammatory effects, and bioactive diversity thereof greatly fits the pathogenesis of DN. Thirdly, both of the two compounds in administration way of solid dispersions can effectively improve the renal function of the rats suffered from the type II diabetic nephropathy, and main treatment indexes thereof, such as uric acid, urinary protein, microalbuminuria, transforming growth factor TGF-β1, blood glucose and low-density lipoprotein cholesterol, are better than that of captopril which is a clinical first-line drug. Fourthly, the two compounds have strong antimicrobial and immunomodulatory effects. Fifthly, until now, there is no report about the pharmacological effects of the two compounds in resisting the type II diabetic nephropathy and treating the acute pyelonephritis and the chronic nephritis at home and abroad.

These and other objectives, features, and advantages of the present invention will become apparent from the following detailed description, the accompanying drawings, and the appended claims.

### BRIEF DESCRIPTION OF THE DRAWINGS

FIG. 1 is a sketch view of kidney pathology comparison among diabetic nephropathy model rats of each group under electron microscope with hematoxylin-eosin (HE) staining (HE×200).

FIG. 2 is a sketch view of kidney pathology comparison among diabetic nephropathy model rats of each group under electron microscope with Masson staining (Masson×400).

FIG. 3 is a sketch view of urinary bacterial culture situation of acute pyelonephritis rats.

FIG. 4 is a sketch view of bacterial culture situation of renal extract of the acute pyelonephritis rats.

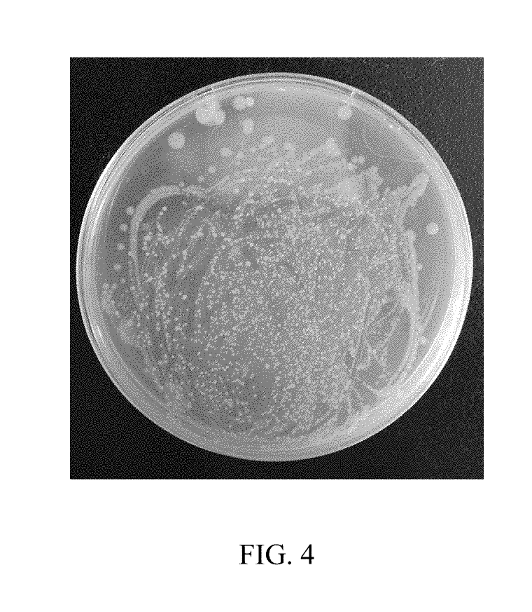

FIG. 5 is a sketch view of kidney pathology comparison among acute pyelonephritis model rats of each group under electron microscope with HE staining (HE×200).

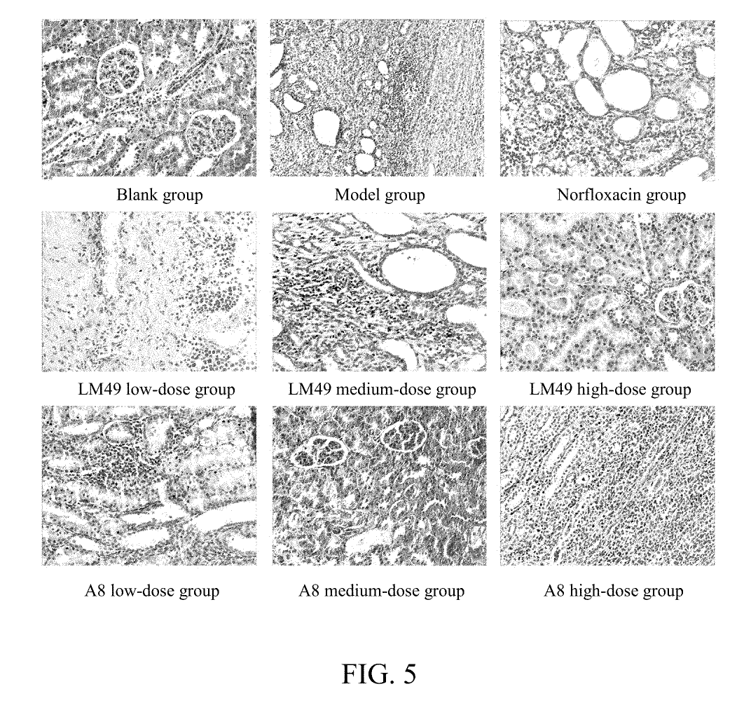

### DETAILED DESCRIPTION OF THE PREFERRED EMBODIMENT

Applications of 2,5′-dibromo-4,5,2′-trihydroxyldiphenylmethanone and 4,5,2′-tris(4-morpholinemethanoyl)-2,5′-dichlorobenzophenone in preparation of drugs respectively for resisting type II diabetic nephropathy and for treating acute pyelonephritis and chronic nephritis are provided.

The above drugs respectively for resisting the type II diabetic nephropathy and for treating the acute pyelonephritis and the chronic nephritis adopt the 2,5′-dibromo-4,5,2′-trihydroxyldiphenylmethanone or the 4,5,2′-tris(4-morpholinemethanoyl)-2,5′-dichlorobenzophenone as an active ingredient, and further comprise an ordinary medicinal carrier or an excipient which is used combined with the active ingredient. According to specific requirements, the drugs can be prepared into common dosage forms, such as injections, tablets, pills, solid dispersions and capsules.

Pharmacological effects of the 2,5′-dibromo-4,5,2′-trihydroxyldiphenylmethanone and the 4,5,2′-tris(4-morpholinemethanoyl)-2,5′-dichlorobenzophenone in resisting the type II diabetic nephropathy and treating the acute pyelonephritis and the chronic nephritis are described as follows with examples. It is noted that the examples are illustrative only, not for limiting the present invention.

### Example 1

Materials:

Experimental Animals:

Male SD (Sprague Dawley) rats having a weight of 180-220 g from National Institutes for Food and Drug Control with a license number of SCXK-(Beijing)2014-0013 and a certification number of 11400500008465 are selected. The used experimental animals and related dispositions thereof meet animal welfare requirements, and an ethical review of animal welfare council is required before experiments.

Diet:

A high-sugar and high-fat diet is selected, comprising components of: 10%0 lard oil; 20% sucrose; 2.5% cholesterol: 0.5% sodium cholate; and 67% basal diet. The diet is from Beijing Keao Xieli Food Co., Ltd. with a license number of SCXK-(Beijing)2014-0010 and a certification number of 11002900016120.

Drugs and Reagents:

The drugs and reagents comprise: 2,5′-dibromo-4,5,2′-trihydroxyldiphenylmethanone (hereafter referred to as LM49, prepared by pharmaceutical chemistry laboratory of Shanxi Medical University, with a purity larger than or equal to 99.5% and a batch number of 20150320); 4,5,2′-tris(4-morpholinemethanoyl)-2,5′-dichlorobenzophenone (hereafter referred to as A8, prepared by pharmaceutical chemistry laboratory of Shanxi Medical University, with a purity larger than or equal to 99.5% and a batch number of 20150112); polyvinylpyrrolidone (PVP K30) (USP26, produced by Jiangyin Jiafeng Chemical Co., Ltd.); tween80 (medical-grade, produced by Sichuan Jinshan Pharmaceutical Co., Ltd.); absolute ethyl alcohol (analytically pure, produced by Beijing Chemical Reagent Co., Ltd.); captopril (produced by Shanghai Xudong Haipu Pharmaceutical Co., Ltd. with a batch number of 140603); streptozotocin (produced by Sigma Co., Ltd. with a batch number of 1126C038); rat interleukin-1β ELISA (enzyme-linked immuno-sorbent assay) kit (produced by Nanjing Jiancheng Technology Co., Ltd. with a batch number of 1501261); rat interleukin-6 ELISA kit (produced by Nanjing Jiancheng Technology Co., Ltd. with a batch number of 1412271); rat microalbumin (mAlb) ELISA kit (produced by Nanjing Jiancheng Technology Co., Ltd. with a batch number of 1506201); rat soluble intercellular adhesion molecule-1 (sICAM-1) assay kit (produced by Nanjing Jiancheng Technology Co., Ltd. with a batch number of 1501201); rat tumor necrosis factor-α (TNF-α) ELISA kit (produced by Nanjing Jiancheng Technology Co., Ltd. with a batch number of 1507281); rat soluble vascular cell adhesion molecule-1 (sVCAM-1) ELISA kit (produced by Nanjing Jiancheng Technology Co., Ltd. with a batch number of 1508221); total antioxidant capacity (T-AOC) assay kit (produced by Nanjing Jiancheng Technology Co., Ltd. with a batch number of 20151010); BCA (bicinchoninic acid) protein assay kit (produced by BOSTER Biological Technology Co., Ltd. with a batch number of 10K12B46); blood urea nitrogen (BUN) assay kit (produced by Shanghai Fosun Long March Medical Science Co., Ltd. with a batch number of P1311021); uric acid (UA) assay kit (produced by Shanghai Fosun Long March Medical Science Co., Ltd. with a batch number of PF1405011); creatinine (CREA) assay kit (produced by Shanghai Fosun Long March Medical Science Co., Ltd. with a batch number of D1 404063); clinical chemical comprehensive quality control serum (produced by Shanghai Fosun Long March Medical Science Co., Ltd. with a batch number of 842UN); malondialdehyde (MDA) assay kit (produced by Nanjing Jiancheng Technology Co., Ltd. with a batch number of 20150126); rat nuclear factor-kappa B (NF-κB) ELISA kit (produced by Nanjing Jiancheng Technology Co., Ltd. with a batch number of 1507251); superoxide dismutase (SOD) assay kit (water-soluble tetrazolium-1 (WST-1) method, produced by Nanjing Jiancheng Technology Co., Ltd. with a batch number of 20150127); and Masson stain (produced by Nanjing Jiancheng Technology Co., Ltd. with a batch number of 20150915).

Instruments:

The instruments comprise: thermostat water bath (produced by Shanghai Hasuc Instrument Manufacture Co., Ltd.); BP-121S electronic balance (produced by Shanghai Precision & Scientific Instrument Co., Ltd.); TU-1810 ultraviolet and visible spectrophotometer (produced by Beijing Purkinje General Instrument Co., Ltd.); glucometer (produced by Johnson & Johnson); microplate reader (produced by Rayto Co., Ltd., RT-6100); low-speed autobalancing centrifuge (produced by Baiyang Centrifuge Factory, Anxin county, Hebei Province); ultra-low temperature freezer (produced by Haier Co., Ltd.); and Konelab Prime 30 fully automatic biochemical analyzer (produced by American Thermo Co., Ltd.).

Preparation of Solid Dispersions:

The solid dispersions are prepared through a solvent method, comprising steps of: respectively precisely weighing LM49, PVPK30 and tween80 with a weight ratio of 1:5:1, and A8, PVPK30 and tween80 with a weight ratio of 1:4:1; dissolving with appropriate absolute ethyl alcohol, continuously stirring, and uniformly mixing; placing on the water bath at 60° C., evaporating and removing the absolute ethyl alcohol, and obtaining a viscous material; placing in the freezer at −20° C., and freezing for 2 hours; taking out from the freezer, then placing in the vacuum drying oven, and drying for 24 hours; after becoming embrittlement, smashing and sieving with a sieve of 80 meshes, and then placing in the dryer for storage.

Preparation of Diabetic Nephropathy (DN) Animal Models:

The healthy adult male SD rats (having a weight of 180-220 g) are fed with the high-sugar and high-fat diet for 4 weeks; the rats are fasting overnight; and streptozocin (STZ) of 40 mg/kg is intraperitoneally injected into the rats. The rats of normal control group are injected with the sodium citrate buffer of same volume; 72 hours later, the rats are fasting for 12 hours. The fasting blood glucose of the model rats is measured; if the fasting blood glucose of the rats is higher than 16.7 mmol/L for consecutive three times, it is considered that the models are successfully established. During the administration period, the successfully established model rats are still fed with the high-sugar and high-fat diet, while the rats of the normal control group are fed with the normal diet.

After administration for 6 weeks, 6-hour urine of the rats is collected with the metabolism cage.

The rats are fasting overnight and anesthetized intraperitoneally, then the abdominal aorta blood thereof is drawn for measuring the related indexes. The two kidneys of each rat are washed with the normal saline; weights of the kidneys are recorded; and the kidney coefficient (weights of two kidneys/body weight) is calculated. The renal tissues (comprising the glomerulus and renal tubules) at the same part of the left kidney are cut lengthwise and immersed in the 10% neutral formalin for pathomorphology analysis, and the other parts of the left kidney are used for biochemical index measurement; the right kidney is used for mechanism research and stored in the freezer at −80° C.

### Groups and Dose Designs:

After successfully establishing the model rats, expect the normal control group (ten rats), the remaining rats are randomly divided into eight groups, wherein each group has twelve rats. The eight groups are respectively the model group, the LM49 solid dispersion low-dose group (with the drug concentration of 3 mg/kg), the LM49 solid dispersion medium-dose group (with the drug concentration of 9 mg/kg), the LM49 solid dispersion high-dose group (with the drug concentration of 27 mg/kg), the A8 solid dispersion low-dose group (with the drug concentration of 3 mg/kg), the A8 solid dispersion medium-dose group (with the drug concentration of 9 mg/kg), the A8 solid dispersion high-dose group (with the drug concentration of 27 mg/kg), and the captopril control group (with the drug concentration of 15 mg/kg).

Administration Method:

Administration Way: By Gavage.

Administration Volume:

The rats of the normal control group and the model group are administrated with the normal saline of 1.0 ml/100 g; the rats of the positive control group, the LM49 solid dispersion high-dose, medium-dose and low-dose groups and the A8 solid dispersion high-dose, medium-dose and low-dose groups are respectively administrated with the drugs of different concentrations, and the administration volume is 1.0 ml/100 g.

Drug Preparation:

The solid dispersions are dissolved by the normal saline; and the rats of the positive control group are administrated with the 0.5% CMC-Na (sodium carboxymethyl cellulose) suspension.

Administration Time:

After finishing model establishment, the rats are administrated.

Administration Frequency:

The rats are administrated seven days a week, for totally six weeks.

Statistical Method:

The IBM SPSS Statistics 20 software is used for data processing, and the data is represented in x̅±s. The one-way analysis of variance (ANOVA) is adopted for comparison among the groups. According to the homogeneity test results of variance, if the variance is equal, the LSD (least significant difference) test is adopted; if the variance is unequal, the Dunnett's T3 test is adopted. The difference of P<0.05 is statistically significant.

Results and Analysis:

(1) Effects of LM49 and A8 on Renal Function of DN Rats

TABLE 1
Effects of LM49 and A8 on renal function of DN rats (x̅ ± s, n = 6)

| | Kidney | | |
|---|---|---|---|
| Group | coefficient (%) | CREA (μmol/L) | BUN (mmol/L) |
| Normal control | 0.59 ± 0.08 | 53.88 ± 1.53 | 4.92 ± 0.72 |
| group | | | |
| Model group | 1.03 ± 0.10^{##} | 56.36 ± 4.53 | 12.21 ± 1.31^{##} |
| LM49 high-dose | 1.00 ± 0.09 | 55.12 ± 4.05 | 10.66 ± 2.52 |
| group | | | |
| LM49 medium- | 1.09 ± 0.14 | 55.25 ± 1.01 | 10.97 ± 3.01 |
| dose group | | | |
| LM49 low-dose | 1.08 ± 0.09 | 54.11 ± 3.50 | 11.59 ± 2.78 |
| group | | | |
| A8 high-dose | 0.86 ± 0.05^{□} | 52.06 ± 1.66 | 10.05 ± 1.61 |
| group | | | |
| A8 medium- | 1.00 ± 0.11 | 53.06 ± 3.75 | 10.07 ± 2.85 |
| dose group | | | |
| A8 low-dose | 1.06 ± 0.05 | 54.16 ± 2.95 | 10.56 ± 1.74 |
| group | | | |
| Positive control | 1.02 ± 0.06 | 52.15 ± 3.40 | 10.91 ± 1.15 |
| group (captopril) | | | |

Note:
compared with the normal control group,
^{##}P < 0.01,
^{#}P < 0.05;
compared with the model group,
^{□□}P < 0.01,
^{□}P < 0.05.

TABLE 2
Effects of LM49 and A8 on UA and TGF-β1 of DN rats
(x̅ ± s, n = 6)

| Group | UA (μmol/L) | TGF-β1 (pg/mgprot) |
|---|---|---|
| Normal control | 29.28 ± 6.81 | 18.12 ± 3.97 |
| group | | |
| Model group | 152.77 ± 17.58^{##} | 115.56 ± 22.87^{##} |
| LM49 high-dose | 29.41 ± 7.23^{□□●●} | 30.58 ± 5.83^{□□▴▴} |
| group | | |
| LM49 medium- | 25.63 ± 5.83^{□□●●} | 53.60 ± 11.60^{□●} |
| dose group | | |
| LM49 low-dose | 26.01 ± 6.54^{□□●●} | 100.46 ± 25.56^{●●} |
| group | | |
| A8 high-dose | 26.44 ± 5.85^{□□●●} | 21.36 ± 4.64^{□□●●▴▴} |
| group | | |
| A8 medium- | 27.18 ± 5.37^{□□●●} | 27.91 ± 5.90^{□□▴▴} |
| dose group | | |
| A8 low-dose | 28.22 ± 7.14^{□□●●} | 59.35 ± 7.34^{□□} |
| group | | |
| Positive control | 49.00 ± 3.24^{□□} | 36.72 ± 11.42^{□□} |
| group (captopril) | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

TABLE 3
Effects of LM49 and A8 on urinary protein and microalbumin in
urine of DN rats (x̅ ± s, n = 6)

| Group | Urinary protein (mg/6 h) | mALB (μg/6 h) |
|---|---|---|
| Normal control | 1.32 ± 0.16 | 34.31 ± 6.04 |
| group | | |
| Model group | 11.02 ± 1.95^{##} | 246.18 ± 45.12^{##} |
| LM49 high-dose | 2.82 ± 0.54^{□□▴◯●} | 52.22 ± 7.46^{□□▴▴} |
| group | | |
| LM49 medium-dose | 5.29 ± 0.61^{□□} | 125.38 ± 23.67^{□□●} |
| group | | |
| LM49 low-dose | 5.81 ± 1.42^{□□} | 143.47 ± 33.28^{□□●●} |
| group | | |
| A8 high-dose group | 1.42 ± 0.71^{□□●●▴} | 39.00 ± 7.58^{□□●●▴▴} |
| A8 medium-dose | 2.80 ± 1.02^{□□●●} | 51.76 ± 9.44^{□□●▴} |
| group | | |
| A8 low-dose group | 3.64 ± 0.65^{□□} | 88.91 ± 10.33^{□□} |
| Positive control | 5.93 ± 1.59^{□□} | 76.69 ± 7.69^{□□} |
| group (captopril) | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

(2) Effects of LM49 and A8 on Blood Glucose Level in Serum of DN Rats

TABLE 4
Effects of LM49 and A8 on blood glucose level in serum of DN rats (x̅ ± s, n = 6)

| | Glycated | Glucose (GLU) |
|---|---|---|
| Group | hemoglobin (%) | (mmol/L) |
| Normal control | 4.00 ± 0.00 | 9.69 ± 1.36 |
| group | | |
| Model group | 9.09 ± 0.72^{##} | 34.05 ± 2.98^{##} |
| LM49 high-dose | 8.10 ± 1.50 | 10.73 ± 2.89^{□□▴◯} |
| group | | |
| LM49 medium-dose | 8.59 ± 0.85 | 18.58 ± 2.71^{□□} |
| group | | |
| LM49 low-dose | 9.01 ± 0.61 | 19.94 ± 6.09^{□□} |
| group | | |
| A8 high-dose group | 6.44 ± 0.53^{□□●} | 9.75 ± 2.11^{□□●●▴◯} |
| A8 medium-dose | 7.33 ± 0.77^{□} | 11.86 ± 2.96^{□□▴} |
| group | | |
| A8 low-dose group | 8.64 ± 0.45 | 13.11 ± 3.95^{□□} |
| Positive control | 8.88 ± 0.55 | 16.66 ± 4.34^{□□} |
| group (captopril) | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

(3) Effects of LM49 and A8 on Total Cholesterol (TCHO), Triglyceride (TG), High-Density Lipoprotein Cholesterol (HDL-C), and Low-Density Lipoprotein Cholesterol (LDL-C) in Serum of DN Rats

TABLE 5
Effects of LM49 and A8 on TC and TG in serum of DN
rats (x̅ ± s, n = 6)

| Group | TC (mmol/L) | TG (mmol/L) |
|---|---|---|
| Normal control group | 1.24 ± 0.23 | 0.53 ± 0.14 |
| Model group | 20.38 ± 2.39^{##} | 1.77 ± 0.43^{#} |
| LM49 high-dose group | 3.00 ± 0.79^{□□} | 0.54 ± 0.13^{□□} |
| LM49 medium-dose group | 2.71 ± 0.49^{□□} | 0.30 ± 0.11^{□□} |
| LM49 low-dose group | 3.28 ± 0.59^{□□} | 0.38 ± 0.10^{□□} |
| A8 high-dose group | 1.42 ± 0.19^{□□●●▴◯} | 0.30 ± 0.08^{□□} |
| A8 medium-dose group | 2.33 ± 0.58^{□□} | 0.30 ± 0.07^{□□} |
| A8 low-dose group | 2.87 ± 0.69^{□□} | 0.39 ± 0.05^{□□} |
| Positive control group | 3.06 ± 0.43^{□□} | 0.37 ± 0.09^{□□} |
| (captopril) | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

TABLE 6
Effects of LM49 and A8 on HDL-C and LDL-C in serum of
DN rats (x̅ ± s, n = 6)

| Group | HDL-C (mmol/L) | LDL-C (mmol/L) |
|---|---|---|
| Normal control group | 1.23 ± 0.16 | 0.22 ± 0.07 |
| Model group | 1.11 ± 0.23 | 13.97 ± 4.82^{##} |
| LM49 high-dose group | 1.17 ± 0.24 | 1.61 ± 0.54^{□□} |
| LM49 medium-dose group | 1.16 ± 0.21 | 1.51 ± 0.48^{□□} |
| LM49 low-dose group | 1.27 ± 0.12 | 1.90 ± 0.54^{□□} |
| A8 high-dose group | 1.20 ± 0.27 | 1.01 ± 0.20^{□□●▴} |
| A8 medium-dose group | 1.23 ± 0.40 | 1.41 ± 0.33^{□□} |
| A8 low-dose group | 1.21 ± 0.28 | 1.77 ± 0.57^{□□} |
| Positive control group | 1.19 ± 0.35 | 1.80 ± 0.27^{□□} |
| (captopril) | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

(4) Effects of LM49 and A8 on Inflammatory Factors in Renal Tissues of DN Rats

TABLE 7
Effects of LM49 and A8 on inflammatory factors of TNF-α, NF-κB and ICAM-1
in renal tissues of DN rats (x̅ ± s, n = 6)

| Group | TNF-α (pg/mgprog) | NF-κB (pg/mgprog) | ICAM-1 (pg/mgprog) |
|---|---|---|---|
| Normal control group | 304.40 ± 65.141 | 293.81 ± 46.31 | 336.03 ± 33.64 |
| Model group | 574.13 ± 104.00^{#} | 2500.670 ± 464.93^{##} | 401.74 ± 95.97 |
| LM49 high-dose group | 279.29 ± 41.89^{□} | 215.39 ± 43.61^{□□▴▴◯◯} | 282.88 ± 59.07 |
| LM49 medium-dose group | 436.04 ± 84.54^{●} | 1017.81 ± 239.12^{□▴●●} | 334.79 ± 55.46 |
| LM49 low-dose group | 573.35 ± 150.75 | 2415.59 ± 515.30^{●●} | 384.02 ± 100.59 |
| A8 high-dose group | 304.51 ± 73.47^{□} | 298.32 ± 58.92^{□□▴▴◯◯} | 322.5 ± 85.70 |
| A8 medium-dose group | 321.27 ± 86.34^{□} | 786.93 ± 79.73^{□□▴▴●} | 321.63 ± 82.35 |
| A8 low-dose group | 485.60 ± 138.80^{●} | 1601.45 ± 206.73^{□●●} | 339.84 ± 92.15 |
| Positive control group | 292.79 ± 81.62^{□} | 174.49 ± 45.87^{□□} | 246.36 ± 49.25 |
| (captopril) | | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

TABLE 8
Effects of LM49 and A8 on inflammatory factors of interleukin-1β (IL-1β),
interleukin-6 (IL-6) and VCAM-1 in renal tissues of DN rats (x̅ ± s, n = 6)

| Group | IL-1β (pg/mgprog) | IL-6 (pg/mgprog) | VCAM-1 (pg/mgprog) |
|---|---|---|---|
| Normal control group | 129.62 ± 11.78 | 69.34 ± 13.17 | 33.02 ± 5.27 |
| Model group | 160.42 ± 21.93 | 195.42 ± 2.34^{##} | 39.34 ± 10.56 |
| LM49 high-dose group | 121.69 ± 16.12 | 119.09 ± 11.51^{□} | 26.85 ± 5.63 |
| LM49 medium-dose group | 146.20 ± 16.12 | 168.44 ± 30.33 | 34.76 ± 5.04 |
| LM49 low-dose group | 158.77 ± 43.38 | 195.42 ± 2.34 | 39.34 ± 10.56 |
| A8 high-dose group | 133.45 ± 24.68 | 71.29 ± 14.14^{□□●} | 30..41 ± 6.71 |
| A8 medium-dose group | 155.22 ± 19.45 | 111.69 ± 35.86^{□} | 31.09 ± 5.42 |
| A8 low-dose group | 178.45 ± 31.59 | 139.85 ± 55.41^{□} | 37.85 ± 9.28 |
| Positive control group | 102.32 ± 22.70 | 107.50 ± 20.22^{□□} | 23.79 ± 3.50 |
| (captopril) | | | |

Note:
compared with the normal control group,
^{##}P < 0.01,
^{#}P < 0.05;
compared with the model group,
^{□□}P < 0.01,
^{□}P < 0.05;
compared with the low-dose groups,
^{▴▴}P < 0.01,
^{▴}P < 0.05;
compared with the medium-dose groups,
^{◯◯}P < 0.01,
^{◯}P < 0.05;
compared with the captopril control group,
^{●●}P < 0.01,
^{●}P < 0.05.

(5) Effects of LM49 and A8 on Oxidant Factors in Renal Tissues of DN Rats

TABLE 9
Effects of LM49 and A8 on oxidant factors in renal tissues of DN rats (x̅ ± s, n = 6)

| Group | T-AOC (U/mgprog) | SOD (U/mgprog) | MDA (nmol/mgprog) |
|---|---|---|---|
| Normal control group | 1.39 ± 0.66 | 5.57 ± 1.75 | 1.42 ± 0.38 |
| Model group | 1.89 ± 0.43 | 4.77 ± 0.69 | 1.75 ± 0.49 |
| LM49 high-dose group | 1.37 ± 0.31 | 4.06 ± 0.78 | 1.16 ± 0.19 |
| LM49 medium-dose group | 1.93 ± 0.43 | 5.33 ± 1.01 | 1.92 ± 0.34 |
| LM49 low-dose group | 1.52 ± 0.58 | 5.66 ± 1.87 | 1.65 ± 0.25 |
| A8 high-dose group | 1.32 ± 0.17 | 4.37 ± 1.25 | 1.42 ± 0.25 |
| A8 medium-dose group | 1.37 ± 0.31 | 5.12 ± 1.41 | 1.48 ± 0.16 |
| A8 low-dose group | 1.39 ± 0.19 | 5.57 ± 1.75 | 1.52 ± 0.38 |
| Positive control group | 1.48 ± 0.41 | 4.42 ± 0.94 | 1.04 ± 0.24 |
| (captopril) | | | |

The above researches show that: the LM49 has the obvious effect on resisting the DN of rats, which is able to effectively reduce the lesions of the renal tissues, obviously decrease the UA, GLU, TC, TG and LDL-C in the serum and the TGF-β1, urinary protein, and microalbuminuria in the renal tissues of the rats, obviously suppress the expressions of the inflammatory factors such as TNF-α, NF-κB, IL-6 and IL-1β in the renal tissues, and decrease the tubular watery lesion and glomerular fibrosis level. Meanwhile, the above results also indicate that: LM49 has the strong effect in resisting the DN of rats; and the main treatment indexes thereof, such as UA, urinary protein, microalbuminuria, TGF-β1, GLU and LDL-C, are better than that of captopril which is a clinical first-line drug, showing the important application prospect.

A8 has the stronger effect in resisting the DN of rats, which is able to effectively reduce the lesions of the renal tissues, obviously decrease the UA, glycated hemoglobin, GLU, TC, TG and LDL-C in the serum and the TGF-β1, urinary protein, and microalbuminuria in the renal tissues of the rats, obviously suppress the expressions of the inflammatory factors such as TNF-α, NF-κB, IL-6 and IL-1β in the renal tissues, and decrease the tubular watery lesion and glomerular fibrosis level. Meanwhile, the above results also indicate that: A8 has the strong effect in resisting the DN of rats; and the main treatment indexes thereof, such as kidney coefficient, UA, urinary protein, microalbuminuria, TGF-β1, GLU and LDL-C, are better than that of captopril which is the clinical first-line drug, showing the important application prospect.

Conclusion:

Through the above experiments, it is illustrated that LM49 and A8 have obvious effects on resisting the type II diabetic nephropathy, especially the high-dose groups have the more obvious effects. The compound A8 has the higher activity in resisting the type II diabetic nephropathy than the compound LM49, showing the good application and development prospect.

### Example 2

(1) Experimental Animals, Instruments and Reagents

Animals: 200 SPF (specified pathogen free) male SD rats having a weight of (200+20) g bought from Beijing Huafukang Bioscience Co., Inc. with an experimental animal license number of SCXK(Beijing)2014-0008 and a certification number of 11003800008312 are selected.

Animal Feeding and Feeding Environment:

A clean constant-temperature environment with an average temperature of 25° C., an average humidity of 37%, and full fresh air of 15-20 times/hour is provided for the rats. The rats experience 12 hours of light and 12 hours of dark a day and take food and water freely.

Animal Diet:

The diet is bought from Beijing Huafukang Bioscience Co., Inc. with the license number of SCXK(Beijing)2014-0008 and the certification number of 11003800008312.

Drugs and Reagents:

The drugs and reagents comprise: LM49 and A8, provided by Shanxi Medical University; norfloxacin capsules (produced by Shanxi Taiyuan Pharmaceutical Co., Ltd. with a batch number of 150401); dexamethasone acetate tablets (produced by Zhejiang Xianju Pharmaceutical Co., Ltd. with a batch number of 141006); Escherichia coli ATCC25922 standard strain (produced by Shanghai Luwei Technology Co., Ltd. with a batch number of A1008B); serum creatinine and urine creatinine ELISA kit (produced by Nanjing Jiancheng Technology Co., Ltd. with a batch number of 20160421); IL-la (produced by Neobioscience Technology Company with a batch number of 160429-009a); IL-1β (produced by Neobioscience Technology Company with a batch number of 160429-007a); IL-6 (produced by Neobioscience Technology Company with a batch number of 160429-003a); IL-10 (produced by Neobioscience Technology Company with a batch number of 160429-004a); MCP-1 (monocyte chemoattractant protein-1) (produced by Neobioscience Technology Company with a batch number of 160429-113a); CXCL-2 (chemokine (C—X—C motif) ligand 2) (produced by BOSTER Biological Technology Co., Ltd. with a batch number of 3771190428); blood routine package (produced by American Drew Scientific Co., Ltd. with a batch number of 6572); anti-Rat CD₄ (produced by eBioscience Co., Ltd. with a batch number of 46-0040); and anti-Rat CD₈-PE (produced by eBioscience Co., Ltd. with a batch number of 12-0084).

Main Instruments:

The main instruments comprise: Synergy H1 microplate reader (produced by American BioTek Instruments, Inc.); LRH-150B biochemical incubator (produced by Guangdong Shaoguan Taihong Medical Instrument Co., Ltd.); Thermo 1300 series Class-H Type-A2 biosafety cabinet (produced by American Thermo Fisher Scientific Co., Ltd.); JK-CC30A bacterial colony counter (produced by Shanghai Precision & Scientific Instrument Co., Ltd.); HEMAVET950FS fully automatic hematology analyzer (produced by American Drew Scientific Co., Ltd.); and CYTOMICS FC500 flow cytometry (produced by American Beckman Coulter, Inc.).

(2) Preparation of Solid Dispersions

The solid dispersions are prepared through a solvent method, comprising steps of: respectively precisely weighing LM49, PVPK30 and tween80 with a weight ratio of 1:5:1, and A8, PVPK30 and tween80 with a weight ratio of 1:4:1; dissolving with appropriate absolute ethyl alcohol, continuously stirring, and uniformly mixing; placing on the water bath at 60° C., evaporating and removing the absolute ethyl alcohol, and obtaining a viscous material; placing in the freezer at −20° C., and freezing for 2 hours; taking out from the freezer, then placing in the vacuum drying oven, and drying for 24 hours; after becoming embrittlement, smashing and sieving with a sieve of 80 meshes, and then placing in the dryer for storage.

(3) Model Preparation and Administration

1) Preparation of Acute Pyelonephritis Model Rats, Grouping, and Administration

Ninety male SD rats having a weight of 180-220 g are selected, wherein: ten rats are selected as a sham-operated group (only being processed with ligation, without injecting with Escherichia coli), and the remaining rats are injected with Escherichia coli ATCC25922 standard strain having a concentration of 10⁸/ml according to the method (disclosed in the Chinese Journal of Laboratory Animal Science, 1998, 8(1): pages 31-34) for preparing the acute pyelonephritis model rats.

After three days, urine of the rats is collected for bacterial culture, and the growth situation of the bacteria is observed. If the result is positive, the model rats are successively established; the unqualified rats are excluded, and the operation is made again for complementing the rats. The successively established model rats are randomly divided into the model group, the norfloxacin group (66.7 mg/kg), the LM49 solid dispersion low-dose group (with the drug concentration of 3 mg/kg), the LM49 solid dispersion medium-dose group (with the drug concentration of 9 mg/kg), the LM49 solid dispersion high-dose group (with the drug concentration of 27 mg/kg), the A8 solid dispersion low-dose group (with the drug concentration of 3 mg/kg), the A8 solid dispersion medium-dose group (with the drug concentration of 9 mg/kg), and the A8 solid dispersion high-dose group (with the drug concentration of 27 mg/kg).

The rats of the sham-operated group and the model group are administrated with the normal saline of same volume by gavage, and the rats of the other groups are respectively administrated with the drugs of the corresponding dose, once a day for consecutive ten days.

2) Preparation of Chronic Nephritis Model Rats, Grouping, and Administration

The passive Heymann nephritis is the classic model for researching the human chronic nephrosis. Thus, the present invention researches the effects of the two disclosed compounds on preventing the chronic nephritis with the model.

Ninety male SD rats having a weight of 180-220 g are selected, wherein: ten rats are selected as a blank group, and the remaining rats are used for preparing the rat Heymann nephritis models according to the method (disclosed in Journal of Proteomics, 2012, 75(3): pages 3866-3876). The successively established model rats are randomly divided into the model group, the positive control group (dexamethasone with a concentration of 0.1 mg/kg), the LM49 solid dispersion low-dose group (with the drug concentration of 3 mg/kg), the LM49 solid dispersion medium-dose group (with the drug concentration of 9 mg/kg), the LM49 solid dispersion high-dose group (with the drug concentration of 27 mg/kg), the A8 solid dispersion low-dose group (with the drug concentration of 3 mg/kg), the A8 solid dispersion medium-dose group (with the drug concentration of 9 mg/kg), and the A8 solid dispersion high-dose group (with the drug concentration of 27 mg/kg).

The rats of the blank group and the model group are administrated with the normal saline of same volume, and the rats of the other groups are respectively administrated with the drugs of the corresponding dose, by gavage, every day from model establishment for four weeks.

(4) Sample Collection and Measurement of Related Indexes

1) Acute Pyelonephritis Model Rats

After finishing administration, urine of the rats of all groups is collected (for observing the bacterial growth situation). The abdominal aorta blood of the rats is drawn (for measuring the blood routine, CD₄/CD₈, urine creatinine and serum creatinine in the serum; and for measuring contents of IL-1α, IL-1β, IL-6, IL-10 and MCP-1 in the serum with the ELISA method). Two kidneys of each rat are cut (wherein a part is homogenated for bacterial culture, and the other part is used for observing the pathomorphology change).

TABLE 10
Scoring standard of bacterial growth situation
of kidney with pour-plate culture method

| Score | Bacterial growth situation |
|---|---|
| 1 | Bacterial colony number <10 |
| 2 | Bacterial colony number >10 |
| 3 | Bacterial colony number >100 |
| 4 | Bacterial colony number >1000 |

2) Chronic Nephritis Model Rats

The blood of the rats of all groups is drawn at the second week and the fourth week from model establishment and 24-hour urine thereof is collected, for respectively measuring the contents of the urinary albumin, serum creatinine (Scr) in the serum, BUN, IL-1α, IL-1β, IL-6 and IL-10.

(5) Statistical Method

The data is represented in mean±(standard deviation) (x̅±s) and processed with the SPSS 19.0 statistical software. The one-way analysis of variance (ANOVA) is adopted for comparison among the groups, and then the LSD-Test is adopted for pairwise comparison. The differences of P<0.05 and P<0.01 are statistically significant.

(6) Results and Analysis

1) Results of Treatment Effects of Two Compounds on Acute Pyelonephritis

TABLE 11
Effects of different dose groups on visceral indexes of acute pyelonephritis rats (x̅ ± s, n = 10)

| | | | | Vesical coefficient |
|---|---|---|---|---|
| Group | Dose (mg · kg⁻¹) | Left kidney (g) | Right kidney (g) | (g/100 g) |
| Sham-operated group | — | 0.38 ± 0.02 | 0.40 ± 0.03 | 0.035 ± 0.009 |
| Model group | — | 0.49 ± 0.06** | 2.26 ± 1.76** | 0.053 ± 0.014** |
| Norfloxacin group | 66.7 | 0.44 ± 0.07 | 0.91 ± 0.63^{Δ} | 0.050 ± 0.011 |
| LM49 | Low-dose group | 0.56 ± 0.09 | 2.52 ± 1.43 | 0.054 ± 0.016 |
| | Medium-dose group | 0.51 ± 0.07 | 1.74 ± 1.12 | 0.049 ± 0.011 |
| | High-dose group | 0.48 ± 0.08 | 1.06 ± 0.94^{Δ} | 0.044 ± 0.017^{#} |
| A8 | Low-dose group | 0.52 ± 0.05 | 1.70 ± 1.38 | 0.049 ± 0.013 |
| | Medium-dose group | 0.49 ± 0.06 | 1.11 ± 1.09^{Δ} | 0.043 ± 0.011^{#} |
| | High-dose group | 0.49 ± 0.07 | 0.68 ± 0.32^{ΔΔ} | 0.039 ± 0.012^{##} |

Note:
compared with the sham-operated group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

TABLE 12
Effects of different dose groups on urinary and renal
bacterial culture situation of acute pyelonephritis
rats (x̅ ± s, n = 10)

| | | Urinary bacterial A | Renal bacterial |
|---|---|---|---|
| Group | Dose (mg · kg⁻¹) | value (OD600 nm) | culture score |
| Sham-operated | — | 0.219 ± 0.0665 | 1.3 ± 0.68 |
| group | | | |
| Model group | — | 0.467 ± 0.147** | 3.9 ± 0.32** |
| Norfloxacin | 66.7 | 0.254 ± 0.082^{ΔΔ} | 1.2 ± 0.42^{ΔΔ} |
| group | | | |
| LM49 | Low-dose | 0.353 ± 0.128 | 3.9 ± 0.32 |
| | group | | |
| | Medium-dose | 0.331 ± 0.100^{Δ} | 3.4 ± 0.52^{Δ} |
| | group | | |
| | High-dose | 0.304 ± 0.109^{Δ} | 1.9 ± 0.57^{ΔΔ} |
| | group | | |
| A8 | Low-dose | 0.332 ± 0.131 | 3.6 ± 0.57 |
| | group | | |
| | Medium-dose | 0.304 ± 0.125^{Δ} | 2.6 ± 0.23^{Δ} |
| | group | | |
| | High-dose | 0.274 ± 0.113^{ΔΔ} | 1.5 ± 0.33^{ΔΔ} |
| | group | | |

Note:
compared with the sham-operated group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

TABLE 13
Effects of different dose groups on blood routine indexes of acute pyelonephritis rats (x̅ ± s, n = 10)

| | Dose | | | | | |
|---|---|---|---|---|---|---|
| Group | (mg · kg⁻¹) | WBC (10⁹/L) | NE (10⁹/L) | LY (10⁹/L) | MO (10⁹/L) | EO (10⁹/L) |
| Sham-operated group | — | 4.98 ± 1.56 | 1.67 ± 0.40 | 2.91 ± 1.18 | 0.32 ± 0.16 | 0.07 ± 0.02 |
| Model group | — | 9.05 ± 3.41** | 3.20 ± 1.56** | 5.32 ± 1.97** | 0.65 ± 0.96 | 0.13 ± 0.04* |
| Norfloxacin group | 66.7 | 7.19 ± 2.36 | 2.15 ± 1.30 | 4.66 ± 1.67 | 0.32 ± 0.10 | 0.06 ± 0.01^{Δ} |
| LM49 | Low-dose group | 7.026 ± 2.80 | 2.51 ± 0.87 | 4.15 ± 1.20 | 0.43 ± 0.28 | 0.10 ± 0.05 |
| | Medium-dose group | 6.89 ± 1.87 | 2.33 ± 1.04 | 3.50 ± 1.45 | 0.38 ± 0.22 | 0.09 ± 0.03 |
| | High-dose group | 6.20 ± 2.20^{Δ} | 2.39 ± 1.73 | 3.86 ± 1.38 | 0.40 ± 0.17 | 0.06 ± 0.03^{Δ} |
| A8 | Low-dose group | 6.026 ± 2.60^{Δ} | 2.47 ± 0.76 | 4.07 ± 1.09 | 0.46 ± 0.29 | 0.11 ± 0.03 |
| | Medium-dose group | 5.87 ± 2.12^{Δ} | 2.41 ± 0.94 | 3.74 ± 1.02 | 0.39 ± 0.19 | 0.08 ± 0.01 |
| | High-dose group | 5.73 ± 1.17^{Δ} | 2.38 ± 0.73 | 3.41 ± 1.10 | 0.33 ± 0.15 | 0.06 ± 0.02^{Δ} |

| | Dose | | | | |
|---|---|---|---|---|---|
| Group | (mg · kg⁻¹) | BA (10⁹/L) | RBC (10¹²/L) | Hb (g/L) | MCV (fl) |
| Sham-operated group | — | 0.017 ± 0.04 | 6.97 ± 0.42 | 157.20 ± 6.12 | 62.67 ± 3.80 |
| Model group | — | 0.047 ± 0.06 | 7.00 ± 3.78 | 139.33 ± 15.59** | 61.27 ± 4.23 |
| Norfloxacin group | 66.7 | 0.007 ± 0.01 | 6.60 ± 0.30 | 142.80 ± 9.07^{Δ} | 64.84 ± 3.72 |
| LM49 | Low-dose group | 0.018 ± 0.04 | 5.91 ± 0.51 | 128.00 ± 12.45 | 64.57 ± 2.34 |
| | Medium-dose group | 0.002 ± 0.001 | 6.16 ± 1.02 | 129.10 ± 15.73 | 65.54 ± 6.19 |
| | High-dose group | 0.004 ± 0.01 | 6.67 ± 0.40 | 142.60 ± 7.56^{Δ} | 64.10 ± 3.10 |
| A8 | Low-dose group | 0.017 ± 0.05 | 5.71 ± 0.48 | 131.00 ± 11.44 | 64.33 ± 2.32 |
| | Medium-dose group | 0.006 ± 0.003 | 6.37 ± 0.62 | 134.11 ± 16.42 | 65.43 ± 6.22 |
| | High-dose group | 0.010 ± 0.04 | 6.97 ± 0.51 | 146.42 ± 17.50^{Δ} | 64.16 ± 3.13 |

| | Dose | | | | |
|---|---|---|---|---|---|
| Group | (mg · kg⁻¹) | MCH (pg) | MCHC (g/L) | PLT (10⁹/L) | MPV (fl) |
| Sham-operated group | — | 22.45 ± 1.05 | 331.8 ± 66.71 | 936.15 ± 208.50 | 5.38 ± 0.91 |
| Model group | — | 22.12 ± 1.64 | 336.2 ± 66.09 | 1154.57 ± 226.91* | 6.33 ± 1.31 |
| Norfloxacin group | 66.7 | 21.63 ± 0.47 | 334.5 ± 17.06 | 1478.89 ± 445.85 | 6.33 ± 0.31 |
| LM49 | Low-dose group | 21.64 ± 0.30 | 335.6 ± 9.80 | 1535.10 ± 398.41^{Δ} | 7.32 ± 0.63 |
| | Medium-dose group | 21.23 ± 2.74 | 324.0 ± 31.88 | 1493.70 ± 290.60^{Δ} | 6.48 ± 1.30 |
| | High-dose group | 21.40 ± 0.82 | 334.1 ± 11.15 | 1313.25 ± 249.90 | 6.41 ± 0.29 |
| A8 | Low-dose group | 22.66 ± 0.36 | 332.3 ± 9.45 | 1542.11 ± 401.39^{Δ} | 6.14 ± 0.57 |
| | Medium-dose group | 22.27 ± 2.68 | 331.2 ± 30.64 | 1471.61 ± 275.21^{Δ} | 6.27 ± 0.65 |
| | High-dose group | 22.45 ± 0.67 | 331.5 ± 10.22 | 1320.25 ± 269.95 | 5.75 ± 0.42 |

Note:
compared with the sham-operated group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

TABLE 14
Effects of different dose groups on CD₄, CD₈ and ratio of CD₄ to CD₈
of acute pyelonephritis rats (x̅ ± s, n = 10)

| Group | Dose (mg · kg⁻¹) | CD₄(%) | CD₈(%) | CD₄/CD₈ |
|---|---|---|---|---|
| Sham-operated | — | 32.44 ± 2.18 | 25.29 ± 2.32 | 1.29 ± 0.13 |
| group | | | | |
| Model group | — | 12.93 ± 5.73** | 15.06 ± 6.29** | 0.89 ± 0.29** |
| Norfloxacin | 66.7 | 34.17 ± 11.65^{ΔΔ} | 18.06 ± 5.16 | 1.94 ± 0.62^{ΔΔ} |
| group | | | | |
| LM49 | Low-dose group | 30.88 ± 9.23^{ΔΔ} | 13.75 ± 6.09 | 2.41 ± 0.61^{ΔΔ} |
| | Medium-dose group | 32.06 ± 9.50^{ΔΔ} | 16.49 ± 4.81 | 1.98 ± 0.39^{ΔΔ} |
| | High-dose group | 32.45 ± 2.15^{ΔΔ} | 25.32 ± 2.52^{ΔΔ##} | 1.29 ± 0.14^{ΔΔ#} |
| A8 | Low-dose group | 31.44 ± 9.66^{ΔΔ} | 17.03 ± 5.89 | 1.86 ± 0.63^{ΔΔ} |
| | Medium-dose group | 33.06 ± 9.50^{ΔΔ} | 20.49 ± 4.56 | 1.47 ± 0.42^{ΔΔ} |
| | High-dose group | 31.13 ± 3.11^{ΔΔ} | 24.32 ± 2.02^{ΔΔ##} | 1.28 ± 0.11^{ΔΔ#} |

Note:
compared with the sham-operated group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

TABLE 15
Effects of different dose groups on contents of Ucr, Scr, IL-1α and IL-1β in serum of acute pyelonephritis rats (x̅ ± s, n = 10)

| Group | Dose (mg · kg⁻¹) | Ucr (μmol/l) | Scr (μmol/l) | IL-1α (pg/ml) | IL-1β (pg/ml) |
|---|---|---|---|---|---|
| Sham-operated group | — | 218.73 ± 161.10 | 46.66 ± 14.57 | 7.10 ± 2.76 | 39.56 ± 11.90 |
| Model group | — | 250.75 ± 187.62 | 66.31 ± 22.32 | 13.60 ± 3.84** | 377.52 ± 71.73** |
| Norfloxacin group | 66.7 | 102.94 ± 133.68 | 56.08 ± 14.62 | 6.16 ± 3.14* | 186.96 ± 71.13^{Δ} |
| LM49 | Low-dose group | 195.06 ± 48.33 | 68.98 ± 22.09 | 17.22 ± 2.12 | 388.44 ± 88.07 |
| | Medium-dose group | 195.07 ± 109.47 | 57.91 ± 19.98 | 10.52 ± 4.18 | 361.22 ± 76.99 |
| | High-dose group | 133.68 ± 64.66 | 45.84 ± 16.16 | 8.86 ± 2.86^{Δ} | 200.30 ± 57.80^{Δ} |
| A8 | Low-dose group | 200.04 ± 47.39 | 60.67 ± 20.01 | 12.89 ± 3.71 | 269.53 ± 87.81 |
| | Medium-dose group | 176.22 ± 99.74 | 50.36 ± 12.64 | 10.97 ± 3.88^{Δ} | 229.29 ± 70.42^{Δ} |
| | High-dose group | 110.44 ± 67.86 | 46.84 ± 15.33 | 7.92 ± 3.22^{ΔΔ} | 189.55 ± 30.53^{Δ} |

Effects of different dose groups on contents of IL-6, IL-10, MCP-1 and CXCL-2 in serum of acute pyelonephritis rats (x̅ ± s, n = 10)

| Group | Dose (mg · kg⁻¹) | IL-6 (pg/ml) | IL-10 (pg/ml) | MCP-1 (pg/ml) | CXCL-2 (pg/ml) |
|---|---|---|---|---|---|
| Sham-operated group | — | 46.91 ± 14.53 | 14.64 ± 3.77 | 1294.51 ± 213.93 | 5.16 ± 1.05 |
| Model group | — | 171.34 ± 80.38** | 34.05 ± 10.90** | 1393.37 ± 217.78 | 8.28 ± 2.09* |
| Norfloxacin group | 66.7 | 41.00 ± 14.69^{ΔΔ} | 17.38 ± 1.79^{ΔΔ} | 1536.26 ± 190.48 | 7.19 ± 2.01 |
| LM49 | Low-dose group | 159.33 ± 48.47 | 57.14 ± 15.11 | 1490.47 ± 108.89 | 7.44 ± 1.54 |
| | Medium-dose group | 166.15 ± 74.51 | 30.83 ± 7.91 | 1533.19 ± 196.89 | 7.91 ± 2.35 |
| | High-dose group | 72.21 ± 15.63^{ΔΔ} | 27.14 ± 4.67^{Δ} | 1770.12 ± 113.49 | 7.95 ± 3.05 |
| A8 | Low-dose group | 121.55 ± 43.75 | 51.50 ± 16.11 | 1542.73 ± 106.44 | 7.11 ± 1.62 |
| | Medium-dose group | 80.37 ± 37.80^{Δ} | 20.21 ± 10.44^{Δ} | 1631.11 ± 199.56 | 7.20 ± 2.51 |
| | High-dose group | 51.92 ± 13.29^{ΔΔ} | 15.35 ± 3.73^{ΔΔ} | 1800.09 ± 116.19 | 7.01 ± 1.72 |

Note:
compared with the sham-operated group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

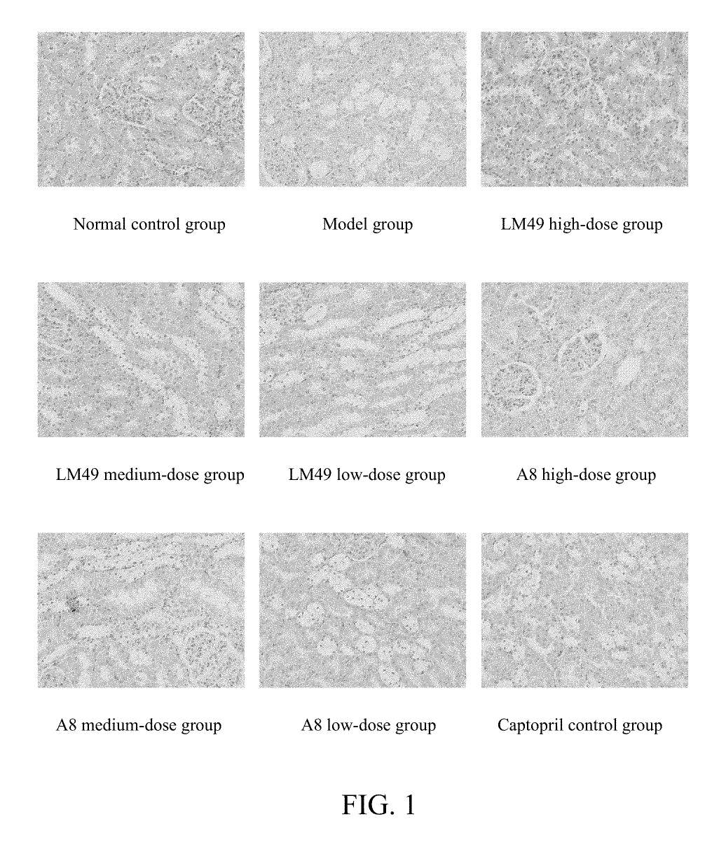

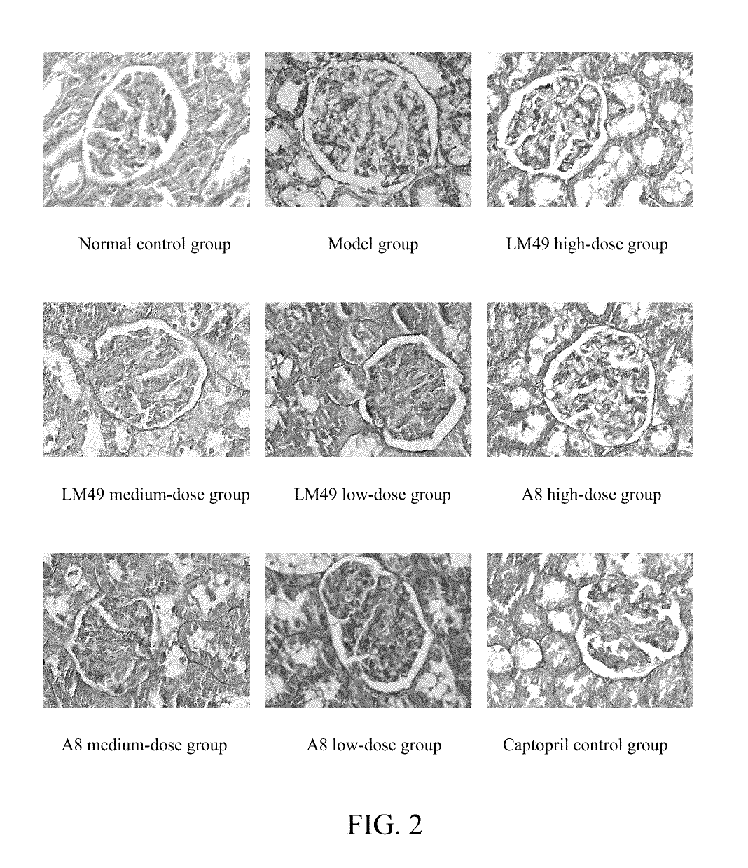

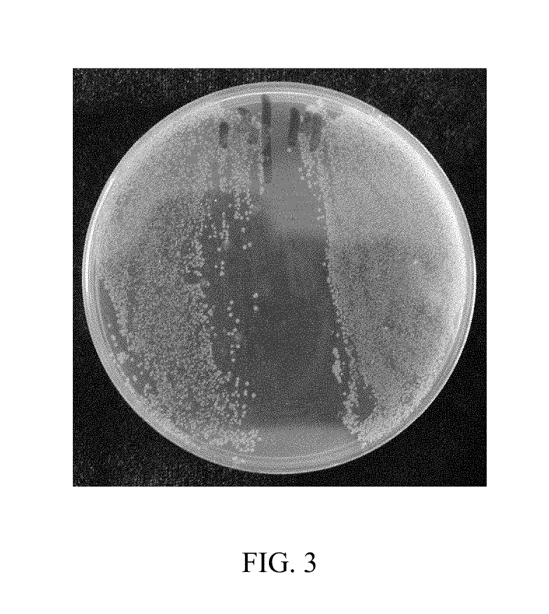

The results show that: the two compounds are able to obviously decrease the kidney coefficient and vesical coefficient of the acute pyelonephritis model rats (referring to Table 2), decrease the bacterial infection level of the renal tissues and urine (referring to Table 12, FIG. 1 and FIG. 2), and obviously decrease the lesions of the cortex and medulla structures, the pelvis and renal calyx tissue structures in the renal tissues, and the chronic inflammatory cell infiltration or abscess (referring to FIG. 3). Moreover, the treatment effects of the two compounds on the acute pyelonephritis is achieved mainly through adjusting the CD₄, CD₈ and CD₄/CD₈ to increase the immunologic function of the model rats, suppressing the expressions of the inflammatory factors such as IL-1α, IL-13 and IL-6, and decreasing the inflammatory reaction. The above results also indicate that: the high-dose groups of LM49 and A8 have the better CD₄/CD adjustment effect than the positive control group, showing the important application prospect.

2) Results of Treatment Effects of Two Compounds on Chronic Nephritis

TABLE 16
Effects of different dose groups on 24-hour urinary protein quantitation in urine
of chronic nephritis rats (x̅ ± s, n = 10)

| | | 24-hour urinary protein quantitation (mg/24 h) | |
|---|---|---|---|
| Group | Dose (mg · kg⁻¹) | Second week | Fourth week |
| Blank group | — | 7.23 ± 1.7 | 6.87 ± 1.5 |
| Model group | — | 33.00 ± 6.1** | 42.33 ± 7.9** |
| Dexamethasone group | 0.1 | 20.87 ± 4.4^{Δ} | 19.99 ± 3.5^{ΔΔ} |
| LM49 | Low-dose group | 34.43 ± 6.6 | 22.18 ± 6.3 |
| | Medium-dose group | 24.99 ± 5.0 | 17.65 ± 5.7^{ΔΔ} |
| | High-dose group | 18.11 ± 3.2^{Δ} | 9.55 ± 2.8^{ΔΔ##} |
| A8 | Low-dose group | 32.98 ± 7.7 | 30.22 ± 6.8 |
| | Medium-dose group | 20.02 ± 5.2^{Δ} | 15.01 ± 4.4^{ΔΔ} |
| | High-dose group | 14.77 ± 3.6^{ΔΔ} | 8.26 ± 1.9^{ΔΔ##} |

Note:
compared with the blank group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

TABLE 17
Effects of different dose groups on Scr and Bun in serum of chronic nephritis rats (x̅ ± s, n = 10)

| | | Scr (μmol/l) | | BUN (μmol/l) | |
|---|---|---|---|---|---|
| Group | Dose (mg · kg⁻¹) | Second week | Fourth week | Second week | Fourth week |
| Blank group | — | 42.01 ± 10.7 | 45.93 ± 10.9 | 8.17 ± 1.1 | 8.77 ± 1.2 |
| Model group | — | 48.78 ± 11.3 | 49.88 ± 12.1 | 8.33 ± 0.9 | 7.99 ± 0.8 |
| Dexamethasone group | 0.1 | 46.22 ± 10.4 | 44.32 ± 13.3 | 8.47 ± 1.0 | 8.22 ± 0.5 |
| LM49 | Low-dose group | 49.44 ± 11.1 | 47.78 ± 11.2 | 7.66 ± 0.7 | 8.18 ± 0.7 |
| | Medium-dose group | 45.88 ± 12.2 | 46.57 ± 10.9 | 7.94 ± 0.8 | 8.65 ± 0.6 |
| | High-dose group | 41.42 ± 10.6 | 46.97 ± 11.6 | 8.34 ± 0.6 | 8.27 ± 0.3 |
| A8 | Low-dose group | 44.33 ± 11.1 | 47.36 ± 13.1 | 8.20 ± 0.7 | 8.98 ± 0.9 |
| | Medium-dose group | 45.66 ± 12.2 | 45.99 ± 14.4 | 7.89 ± 0.8 | 7.88 ± 1.4 |
| | High-dose group | 41.04 ± 10.1 | 44.55 ± 10.7 | 8.51 ± 0.6 | 8.38 ± 1.1 |

Note:
compared with the blank group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

TABLE 18
Effects of different dose groups on contents of IL-1α and IL-1β in serum of chronic nephritis rats (x̅ ± s, n = 10)

| | | IL-1α (pg/ml) | | IL-1β (pg/ml) | |
|---|---|---|---|---|---|
| Group | Dose (mg · kg⁻¹) | Second week | Fourth week | Second week | Fourth week |
| Blank group | — | 7.66 ± 2.12 | 7.24 ± 2.05 | 40.31 ± 11.90 | 47.44 ± 10.08 |
| Model group | — | 13.33 ± 3.24** | 14.65 ± 4.35** | 392.55 ± 80.31** | 409.32 ± 89.99** |
| Dexamethasone group | 0.1 | 10.47 ± 2.45^{Δ} | 9.33 ± 4.22^{Δ} | 184.55 ± 57.22^{Δ} | 163.44 ± 59.31^{Δ} |
| LM49 | Low-dose group | 14.53 ± 4.76 | 13.98 ± 3.77 | 388.25 ± 72.31 | 380.39 ± 80.66 |
| | Medium-dose group | 13.62 ± 4.87 | 12.15 ± 4.76 | 351.11 ± 100.32 | 297.55 ± 94.66^{Δ} |
| | High-dose group | 11.44 ± 3.95 | 8.42 ± 2.76^{ΔΔ} | 227.21 ± 95.41^{Δ} | 200.44 ± 59.31^{Δ} |
| A8 | Low-dose group | 14.01 ± 3.99 | 12.65 ± 3.14 | 310.21 ± 70.01 | 261.33 ± 76.55 |
| | Medium-dose group | 12.63 ± 4.56 | 10.10 ± 4.01 | 277.65 ± 60.39 | 192.56 ± 64.69^{Δ} |
| | High-dose group | 10.69 ± 3.76 | 7.33 ± 2.14^{ΔΔ} | 198.20 ± 90.01^{Δ} | 99.44 ± 39.74^{ΔΔ#} |

Effects of different dose groups on contents of IL-6 and IL-10 in serum of chronic nephritis rats (x̅ ± s, n = 10)

| | | IL-6 (pg/ml) | | IL-10 (pg/ml) | |
|---|---|---|---|---|---|
| Group | Dose (mg · kg⁻¹) | Second week | Fourth week | Second week | Fourth week |
| Blank group | — | 53.21 ± 15.13 | 57.64 ± 13.72 | 20.07 ± 6.88 | 24.32 ± 6.05 |
| Model group | — | 180.34 ± 55.30** | 195.01 ± 66.16** | 60.21 ± 19.66** | 71.35 ± 24.92** |
| Dexamethasone group | 0.1 | 87.01 ± 31.44^{ΔΔ} | 61.31 ± 25.66^{ΔΔ} | 51.09 ± 7.55 | 29.44 ± 59.31^{ΔΔ} |
| LM49 | Low-dose group | 131.23 ± 41.33 | 130.12 ± 45.19 | 80.09 ± 33.11 | 60.01 ± 26.61 |
| | Medium-dose group | 100.15 ± 32.51 | 90.66 ± 40.03^{Δ} | 61.31 ± 23.89 | 51.51 ± 18.09 |
| | High-dose group | 91.11 ± 40.63^{ΔΔ} | 77.03 ± 36.53^{ΔΔ} | 55.75 ± 17.61 | 31.42 ± 10.09 |
| A8 | Low-dose group | 111.79 ± 36.28 | 89.33 ± 29.89^{Δ} | 89.33 ± 36.77 | 70.66 ± 32.28 |
| | Medium-dose group | 90.31 ± 30.41^{Δ} | 73.57 ± 25.60^{ΔΔ} | 74.55 ± 32.12 | 61.48 ± 24.53 |
| | High-dose group | 78.66 ± 35.92^{ΔΔ} | 56.95 ± 20.44^{ΔΔ} | 60.25 ± 22.34 | 40.21 ± 13.01 |

Note:
compared with the blank group:
**P < 0.01,
*P < 0.05;
compared with the model group:
^{ΔΔ}P < 0.01,
^{Δ}P < 0.05;
compared with the positive control group:
^{##}P < 0.01,
^{#}P < 0.05.

The results indicate that: both of the two compounds are able to obviously decrease the urinary protein amount and obviously improve the renal function. The effects of the high-dose groups thereof are obviously better than that of the positive control group (referring to Table 16). Moreover, the treatment effects of the two compounds on the chronic nephritis are achieved through suppressing the expressions of the inflammatory factors such as IL-1α, IL-1β and IL-6 to decrease the inflammatory reaction.

Conclusion:

The two compounds have the obvious treatment effects on the acute pyelonephritis model rats and the chronic nephritis model rats, especially the high-dose groups thereof have the more obvious effects, showing the good application and development prospect.

1-8. (canceled)

9: A method for treating type II diabetic nephropathy, acute pyelonephritis or chronic nephritis in a subject, comprising: administrating a composition which comprises a therapeutically effective amount of 2,5′-dibromo-4,5,2′-trihydroxyldiphenylmethanone or 4,5,2′-tris(4-morpholinemethanoyl)-2,5′-dichlorobenzophenone to the subject.

10: A drug for resisting type II diabetic nephropathy, wherein an active ingredient of the drug is 2,5′-dibromo-4,5,2′-trihydroxyldiphenylmethanone or 4,5,2′-tris(4-morpholinemethanoyl)-2,5′-dichlorobenzophenone.

11: The drug for resisting the type II diabetic nephropathy, as recited in claim 10, wherein the drug further comprises a medicinal carrier or an excipient which is used combined with the active ingredient.

12: The drug for resisting the type II diabetic nephropathy, as recited in claim 10, wherein the drug is prepared into injections, tablets, pills, solid dispersions or capsules.

13: The drug for resisting the type II diabetic nephropathy, as recited in claim 11, wherein the drug is prepared into injections, tablets, pills, solid dispersions or capsules.

14: A drug for treating acute pyelonephritis and chronic nephritis, wherein an active ingredient of the drug is 2,5′-dibromo-4,5,2′-trihydroxyldiphenylmethanone or 4,5,2′-tris(4-morpholinemethanoyl)-2,5′-dichlorobenzophenone.

15: The drug for treating the acute pyelonephritis and the chronic nephritis, as recited in claim 14, wherein the drug further comprises a medicinal carrier or an excipient which is used combined with the active ingredient.

16: The drug for treating the acute pyelonephritis and the chronic nephritis, as recited in claim 14, wherein the drug is prepared into solid dispersions, tablets, pills, capsules or injections.

17: The drug for treating the acute pyelonephritis and the chronic nephritis, as recited in claim 15, wherein the drug is prepared into solid dispersions, tablets, pills, capsules or injections.
